(19) United States
(12) Patent Application Publication
(10) Pub. No.: US 2015/0157675 A1
(43) Pub. Date: Jun. 11, 2015

(54) COMPOSITION COMPRISING EUPATORIUM SPP. EXTRACT AS ACTIVE INGREDIENT FOR PREVENTING AND TREATING OBESITY AND METABOLIC BONE DISEASE

(71) Applicants: Hyun Seok Kim, Seoul (KR); Ki Moon Park, Seongnam-si (KR); Min Ji Kim, Seoul (KR); Youngmin Lee, Seoul (KR); Haeng Ran Kim, Seoul (KR); Kang Jin Cho, Yongin-si (KR)

(72) Inventors: Hyun Seok Kim, Seoul (KR); Ki Moon Park, Seongnam-si (KR); Min Ji Kim, Seoul (KR); Youngmin Lee, Seoul (KR); Haeng Ran Kim, Seoul (KR); Kang Jin Cho, Yongin-si (KR)

(73) Assignees: SUNG KYUN BIOTECH CO., LTD., Ansan-si, Gyeonggi-do (KR); REPUBLIC OF KOREA (MANAGEMENT:RURAL DEVELOPMENT ADMINISTRATION), Suwon-si, Gyeonggi-do (KR)

(21) Appl. No.: 14/389,957

(22) PCT Filed: Apr. 2, 2012
(86) PCT No.: PCT/KR2012/002443

§ 371 Date: Jan. 8, 2015

(51) Int. Cl. A61K 36/28; A23L 2/52; A61K 9/48; A23L 1/30; A61K 9/14; A61K 9/20

(52) U.S. Cl. CPC A61K 36/28; A61K 9/14; A61K 9/20; A61K 9/48; A23L 1/3002; A23L 2/52; A23V 2002/00

(57) ABSTRACT

The present invention relates to a Eupatorium spp. extract having anti-obesity effects as a result of decreasing adipocytes and increasing osteoblasts, as well as the effects of preventing bone disease or fractures by increasing osteoblasts and of preventing osteoporosis by decreasing adipocytes and increasing osteoblasts by the same proportion in mesenchymal stem cells. DCM fraction layers of a Eupatorium spp. stem extract collected on a monthly basis and Eupatorium spp. stem extract collected only in September may inhibit the activity of PPARγ, AP2, CD36, adiponectin C/EBPα, and LPL, which serve as significant factors for adipocyte differentiation in C3H10T1/2 cells and primary mesenchymal stem cells, which are pluripotent stem cell lines, and may increase the activity of ALP, osterix, CO1I and RUNX2, which serve as significant factors for osteoblast differentiation. The Eupatorium spp. extract of the present invention exhibits the effects of increasing bone mineral density (BMD) and decreasing adipocytes in bone marrow in an osteoporosis animal model experiment involving an ovariectomy, and therefore may be used as a useful material for preventing and treating osteoporosis.

### TECHNICAL FIELD

The present invention relates to a composition for preventing and treating obesity and metabolic bone diseases, including a Eupatorium spp. extract as an active ingredient. The composition of the present invention can be used to produce health functional foods and pharmaceutical drugs for preventing and treating obesity and metabolic bone diseases.

### BACKGROUND ART

Osteoporosis refers to a condition characterized by low bone mass and deterioration of bone tissue that may lead to weak bones, thus tending to cause bone fracture. Osteoporosis is particularly common in post-menopausal women and is a disease caused by low estrogen levels leading to a remarkable reduction in bone mass. The degree of reduction of bone mass is dependent on differences between individuals or various other factors. When the mass of bones drops morbidly and excessively below a predetermined value, the bones are prone to fracture even by weak shocks. Osteoporosis increases the risk of bone weakness, causing various bone fractures, particularly, thighbone fracture and spinal fracture. Such fractures limit the physical activity of patients with osteoporosis for a long period of time rather than the symptoms of osteoporosis and make it impossible for the patients to enjoy their healthy life. Osteoporosis is known to account for 15% of elderly mortality.

Various substances, including endogenous regulatory factors, are being investigated for the purpose of treating or preventing bone diseases caused by excessive activity of osteoclasts. Drugs currently in use for this purpose exhibit particular pharmacological actions but are known to have several side effects and difficulties in taking medications. Under these circumstances, there is a need to develop novel substances that possess new actions and drug structures, have less toxicity and fewer adverse effects, and are effective in preventing or treating osteoporosis. Since there is a high possibility that such novel substances will be found in natural products that have been used in folk remedies from the past and proven to be non-toxic, considerable attempts have been made to discover and develop new drugs from natural products.

An imbalance between bone-resorbing osteoclasts and bone-forming osteoblasts has been the focus of previous studies on osteoporosis. Further research is being conducted based on the fact that osteoporosis is strongly influenced by bone marrow-derived adipocytes as well as bone-related cells.

Most bone mass is acquired between the ages of 12 to 18 and is affected by hormones and environmental factors during osteogenesis. In this adolescent period, changes in hormone sequence or promotion of bone resorption decreases the mass of bones and increases the risk of bone fractures. Importantly, fractures in youth are associated with changes in bone structure due to skeletal insufficiency and changes in body composition leading to obesity. Bone loss attributed to aging has a significant influence on the relation between fat and bone. Infiltration of bone marrow by fat is a common feature of aging. Both osteoblasts and adipocytes have a common progenitor and are derived from mesenchymal stem cells. As the differentiation of osteoblasts from mesenchymal stem cells decreases, the differentiation of adipocytes from mesenchymal stem cells increases, and aging proceeds, the number of adipocytes in bone marrow increases.

The formation of osteoblasts involves the inhibition of adipocytes to suppress the formation of adipocytes or promote the conversion of existing adipocytes into osteoblasts. This becomes a target for the prevention and treatment of osteoporosis. Conclusively, the relation between fat and bone assists in a pathophysiological understanding of aging-related bone loss and provides a new approach to the treatment and diagnosis of osteoporosis.

Accordingly, it has been recognized that the relation between osteoblasts and adipocytes can be used as a target for the treatment and prevention of age-related osteoporosis. Many efforts have been made to find substances capable of increasing the differentiation of osteoblasts and lowering the differentiation of adipocytes and to develop functional foods using the substances. Particularly, such substances are considered more important new materials for foods/drugs because they exhibit anti-obesity effects and can directly act against bone diseases such as bone fractures to increase the differentiation of osteocytes.

It is known that Eupatorium japonicum, Eupatorium lindleyanum, Eupatorium makinoi var. oppisitifolium, and Eupatorium rugosum belonging to Eupatorium spp. are widely distributed in Korea. Eupatorium spp. are rhizocarpous perennial plants that grow well in humid meadows near the water and are used for edible, ornamental or medicinal applications. Eupatorium spp. have the efficacies of relieving exterior syndrome by means of diaphoresis, expelling cold, and promoting eruption, and have been used to treat prolapse of the anus, measles without rash, rheumatic backache, and cold-related coughs. However, little is known about the effects of Eupatorium spp. against obesity and metabolic bone diseases, including osteoporosis.

### DISCLOSURE

### Technical Problem

It is an object of the present invention to provide a composition for preventing and treating obesity and metabolic bone diseases including an active ingredient derived from a non-toxic natural product, and a health functional food and a pharmaceutical drug produced from the composition.

### Technical Solution

The present invention provides a composition for preventing and treating obesity and metabolic bone diseases, including a Eupatorium spp. extract as an active ingredient.

The present invention also provides a health functional food for preventing and ameliorating obesity and metabolic bone diseases, including a Eupatorium spp. extract as an active ingredient.

The Eupatorium spp. plant is selected from E. japonicum, E. lindleyanum, E. makinoi var. oppisitifolium, E. rugosum, and closely related plants belonging to the genus Eupatorium. E. japonicum is preferred. The Eupatorium spp. extract may be prepared from the whole plant, leaves, stems or flowers of Eupatorium japonicum. Preferably, Eupatorium spp. plants harvested from July to September are excellent in activity taking into consideration the climate of Korea.

As used herein, the extract is defined to include extracts prepared by dissolving Eupatorium spp. plants in a solvent selected from water, including purified water, a C₁-C₄ lower alcohol, a non-polar solvent, and a mixed solvent thereof.

The present invention will now be described in detail.

The Eupatorium spp. extract can be prepared by the following procedure.

First, the whole plant, stems or flowers of a Eupatorium spp. plant are dried in the shade, triturated, and extracted with at least one solvent selected from water, a C₁-C₄ lower alcohol, and a non-polar solvent. The solvent is used in an amount about 1 to about 50 times, preferably about 10 to about 40 times, the weight of the dried sample. The extraction is performed at a temperature of 20 to 110° C., preferably 80 to 100° C., for about 1 to about 6 hours, preferably 2 to 4 hours. Examples of suitable extraction methods include stirring extraction, hot water extraction, cold dipping extraction, reflux cooling extraction, ultrasonic extraction, and supercritical extraction. Preferably, the dried sample is extracted with hot water, filtered, and concentrated or dried under reduced pressure to obtain the crude drug extract.

The non-polar solvent may be dichloromethane, chloroform, diethyl ether, ethyl acetate, hexane, a supercritical fluid, or a mixture thereof.

The mixed solvent may be an aqueous alcoholic solution. In this case, water and the lower alcohol are mixed in a volume ratio of 95:5 to 0.1:99.9. Preferably, a 70 to 99.9% (v/v) aqueous methanolic or ethanolic solution is used as the solvent. The dried sample is extracted with the aqueous methanolic or ethanolic solution and is further fractionated with hexane. The hexane fraction is further fractionated with dichloromethane. The solvent fractions have superior stimulatory activities on the differentiation of osteoblasts and superior inhibitory activities on the differentiation of adipocytes.

The composition of the present invention exhibits a stimulatory activity on the differentiation of osteoblasts and an inhibitory activity on the differentiation of adipocytes. The Eupatorium spp. extract is included in an amount ranging from 0.1 to 50% by weight, based on the total weight of the composition.

The content of the Eupatorium spp. extract is not necessarily limited to the range defined above and may vary depending on the condition of patients in need of treatment and the type and severity of diseases to be treated.

The composition of the present invention may further include one or more suitable carriers, excipients, and diluents that are commonly used for the preparation of pharmaceutical compositions.

The composition of the present invention can be formulated into oral preparations, such as powders, granules, tablets, capsules, suspensions, emulsions, syrups, and aerosols, external preparations, suppositories, and sterile injectable solutions. As the carriers, excipients, and diluents, there may be exemplified lactose, dextrose, sucrose, sorbitol, mannitol, xylitol, erythritol, maltitol, starch, gum acacia, alginate, gelatin, calcium phosphate, calcium silicate, cellulose, methyl cellulose, amorphous cellulose, polyvinyl pyrrolidone, water, methyl hydroxybenzoate, propyl hydroxybenzoate, talc, magnesium stearate, and mineral oil. Each of the preparations may be formulated using a suitable diluent or excipient known in the art, for example, a filler, an extender, a binder, a wetting agent, a disintegrant, or a surfactant. Examples of solid preparations for oral administration include tablets, pills, powders, granules, and capsules. Such solid preparations may be produced by mixing with one or more excipients, such as starch, calcium carbonate, sucrose, lactose, and gelatin. In addition to the simple excipients, there may also be used lubricants, for example, magnesium stearate and talc. Liquid preparations for oral administrations are suspensions, solutions for internal use, emulsions, and syrups. The liquid preparations may include various excipients, for example, wetting agents, sweeteners, aromatic substances, and preservatives, as well as simple diluents known in the art, such as water and liquid paraffin. Sterilized aqueous solutions, non-aqueous solvents, suspensions, emulsions, lyophilizates, and suppositories are included in preparations for parenteral administration. As the non-aqueous solvents and suspensions, there may be used, for example, propylene glycol, polyethylene glycol, vegetable oils such as olive oil, and injectable esters such as ethyl oleate. As bases of the suppositories, there may be used, for example, Witepsol, Macrogol, Tween 61, cacao butter, laurin butter, and glycerogelatin.

A preferred dose of the Eupatorium spp. extract may vary depending on the condition and body weight of patient, the severity of disease, the form of drug, and the route and period of time of administration but can be determined by one skilled in the art. For the desired effects, the Eupatorium spp. extract may be administered in a daily dose of 0.01 mg/kg to 10 g/kg body weight, preferably 1 mg/kg to 1 g/kg body weight, and can be administered in a single dose or in divided doses per day. Accordingly, the dose is not in no way intended to limit the scope of the invention.

The composition of the present invention may be administered to mammals, including rats, mice, livestock, and humans, via various routes. All routes of administration may be contemplated. Examples of suitable administration routes include oral, rectal, intravenous, intramuscular, subcutaneous, intrathecal, epidural, and intracerebroventricular injections.

The present invention provides a health functional food for preventing and ameliorating obesity and metabolic bone diseases, including a Eupatorium spp. extract exhibiting a stimulatory activity on osteoblast differentiation and an inhibitory activity on adipocyte differentiation, and a sitologically acceptable food supplement additive.

For example, the health functional food of the present invention may be selected from various food products, chewing gums, teas, vitamin complexes, and health supplement foods. The health functional food of the present invention may take the form of a powder, a granule, a tablet, a capsule, or a beverage.

The Eupatorium spp. extract is substantially free from toxicity and side effects. Accordingly, the health functional food of the present invention can be taken for a long period of time for prophylactic purposes without fear of toxicity and unexpected side effects.

The Eupatorium spp. extract may be added to a food or beverage to prepare a health food or beverage composition for preventing or ameliorating obesity and metabolic bone diseases. In this case, the extract may be added in an amount of 0.01 to 15% by weight, based on the total amount of the health food composition, and in an amount of 0.02 to 10 g, preferably 0.3 to 1 g, based on 100 ml of the health beverage composition.

The health beverage composition includes a liquid ingredient, in addition to the indicated amount of the Eupatorium spp. extract as an essential ingredient. There is no restriction on the kind of the liquid ingredient. Like general beverages, the health beverage composition of the present invention may further contain various flavoring agents or natural carbohydrates. Examples of the natural carbohydrates include monosaccharides such as glucose and fructose, disaccharides such as maltose and sucrose, polysaccharides such as dextrin and cyclodextrin, general sugars, and sugar alcohols, such as xylitol, sorbitol, and erythritol. As the flavoring agents, there may be advantageously used natural flavoring agents and synthetic flavoring agents. Examples of the natural flavoring agents include thaumatin and stevia extracts (e.g., rebaudioside A and glycyrrhizin. Examples of the synthetic flavoring agents include saccharin and aspartame. The amount of the natural carbohydrates is typically from about 1 to about 20 g, preferably from about 5 to about 12 g, per 100 ml of the composition.

In addition to the foregoing ingredients, the health functional food of the present invention may further contain a variety of nutritional supplements, vitamins, minerals (electrolytes), flavors such as synthetic and natural flavors, colorants, fillers (cheese, chocolate, etc.), pectic acid and salts thereof, alginic acid and salts thereof, organic acids, protective colloids, thickeners, pH-adjusting agents, stabilizers, preservatives, glycerin, alcohols, carbonating agents for carbonated drinks, and the like. The health functional food of the present invention may further contain a flesh to make a natural fruit juice, a fruit juice drink, and a vegetable drink. These ingredients may be used individually or in combination. The total amount of these additives is not important but is generally selected in the range of 0 to 20 parts by weight, based on 100 parts by weight of the composition.

### Advantageous Effects

The Eupatorium spp. extract inhibits the activities of PPARγ, AP2, CD36, adiponectin, C/EBPα, and LPL as adipocyte differentiation-related genes and increases the activities of ALP, osterix, and RUNX2 as osteoblast differentiation-related genes. In addition, the Eupatorium spp. extract increases bone mineral density (BMD) and decreases the number of adipocytes in bone marrow in animal models with osteoporosis caused by ovariectomy. Therefore, the Eupatorium spp. extract can be used to produce health functional foods and pharmaceutical drugs that are effective in preventing, ameliorating, and treating osteoporosis caused by obesity- and age-related bone loss.

### DESCRIPTION OF DRAWINGS

FIG. 1 is a photograph of Eupatorium japonicum before harvest from Mt. Gamak located in Yangju-si, Kyeonggi-do, Korea.

FIG. 2 shows influences of extracts from the whole plant and different sections of Eupatorium japonicum on ALP activity after treatment of C3H10T1/2 cell line with the extracts in osteocyte differentiation media for 9 days.

FIG. 3 graphically shows the relative mRNA expression levels of osteoblast differentiation-related genes after treatment of C3H10T1/2 cell line with an extract from the whole plant of Eupatorium japonicum in osteocyte differentiation media for 10 days.

FIG. 4 graphically shows the relative mRNA expression levels of osteoblast differentiation-related genes after treatment of C3H10T1/2 cell line with an extract from the leaves of Eupatorium japonicum in osteocyte differentiation media for 9 days.

FIG. 5 graphically shows the relative mRNA expression levels of osteoblast differentiation-related genes after treatment of C3H10T1/2 cell line with an extract from the stems of Eupatorium japonicum in osteocyte differentiation media for 9 days.

FIG. 6 graphically shows the relative mRNA expression levels of osteoblast differentiation-related genes after treatment of C3H10T1/2 cell line with an extract from the flowers of Eupatorium japonicum in osteocyte differentiation media for 9 days.

FIG. 7 shows inhibitory effects of extracts from the whole plant and different sections of Eupatorium japonicum on adipocyte differentiation after treatment of C3H10T1/2 cell line with the extracts in adipocyte differentiation media for 9 days.

FIG. 8 graphically shows the relative mRNA expression levels of adipocyte differentiation-related genes after treatment of C3H10T1/2 cell line with an extract from the whole plant of Eupatorium japonicum in adipocyte differentiation media for 10 days.

FIG. 9 graphically shows the relative mRNA expression levels of adipocyte differentiation-related genes after treatment of C3H10T1/2 cell line with an extract from the leaves of Eupatorium japonicum in adipocyte differentiation media for 10 days.

FIG. 10 graphically shows the relative mRNA expression levels of adipocyte differentiation-related genes after treatment of C3H10T1/2 cell line with an extract from the stems of Eupatorium japonicum in adipocyte differentiation media for 10 days.

FIG. 11 graphically shows the relative mRNA expression levels of adipocyte differentiation-related genes after treatment of C3H10T1/2 cell line with an extract from the flowers of Eupatorium japonicum in adipocyte differentiation media for 10 days.

FIG. 12 shows photographs of osteocyte differentiation media in which C3H10T1/2 cell line was treated with extracts from the stems of Eupatorium japonicum harvested in different months for 9 days, followed by ALP staining.

FIG. 13 shows photographs of osteocyte differentiation media in which C3H10T1/2 cell line was treated with an extract from the stems of Eupatorium japonicum for 9 days, followed by ALP staining.

FIG. 14 shows photographs of osteocyte differentiation media in which primary mesenchymal stem cells were treated with an extract from the stems of Eupatorium japonicum for 9 days, followed by ALP staining.

FIG. 15 shows photographs of osteocyte differentiation media in which C3H10T1/2 cell line was treated with six solvent fractions from an extract from the stems of Eupatorium japonicum harvested in September for 9 days, followed by ALP staining.

FIG. 16 shows photographs of osteocyte differentiation media in which C3H10T1/2 cell line was treated with a DCM fraction from an extract from the stems of Eupatorium japonicum for 9 days, followed by ALP staining.

FIG. 17 graphically shows the relative mRNA expression levels of osteoblast differentiation-related genes after treatment of C3H10T1/2 cell line with an extract from the stems of Eupatorium japonicum in osteocyte differentiation media for 9 days.

FIG. 18 graphically shows the relative mRNA expression levels of osteoblast differentiation-related genes after treatment of primary mesenchymal stem cells with an extract from the stems of Eupatorium japonicum in osteocyte differentiation media for 9 days.

FIG. 19 graphically shows the relative mRNA expression levels of osteoblast differentiation-related genes after treatment of C3H10T1/2 cell line with a DCM fraction from an extract from the stems of Eupatorium japonicum in osteocyte differentiation media for 9 days.

FIG. 20 shows photographs of adipocyte differentiation media in which C3H10T1/2 cell line was treated with extracts from the stems of Eupatorium japonicum harvested in different months for 9 days, followed by Oil Red 0 staining.

FIG. 21 shows photographs of adipocyte differentiation media in which C3H10T1/2 cell line was treated with an extract from the stems of Eupatorium japonicum for 9 days, followed by Oil Red 0 staining.

FIG. 22 shows photographs of adipocyte differentiation media in which C3H10T1/2 cell line was treated with six solvent fractions from an extract from the stems of Eupatorium japonicum harvested in September for 9 days, followed by Oil Red O staining.

FIG. 23 shows photographs of adipocyte differentiation media in which C3H10T1/2 cell line was treated with a DCM fraction from an extract from the stems of Eupatorium japonicum for 9 days, followed by Oil Red 0 staining.

FIG. 24 graphically shows the relative mRNA expression levels of adipocyte differentiation-related genes after treatment of C3H10T1/2 cell line with an extract from the stems of Eupatorium japonicum in adipocyte differentiation media for 9 days.

FIG. 25 graphically shows the relative mRNA expression levels of adipocyte differentiation-related genes after treatment of primary mesenchymal stem cells with an extract from the stems of Eupatorium japonicum in adipocyte differentiation media for 9 days.

FIG. 26 graphically shows the relative mRNA expression levels of adipocyte differentiation-related genes after treatment of C3H10T1/2 cell line with a DCM fraction from an extract from the stems of Eupatorium japonicum in adipocyte differentiation media for 9 days.

FIG. 27 graphically shows changes in the body weight of ovariectomized white rat models after treatment with an extract from the stems of Eupatorium japonicum harvested in September.

FIG. 28 graphically shows changes in the bone mineral density (BMD) of ovariectomized white rat models after treatment with an extract from the stems of Eupatorium japonicum harvested in September.

FIG. 29 shows the results of histological analysis for ovariectomized white rat models after treatment with an extract from the stems of Eupatorium japonicum harvested in September, followed by H&E staining.

### BEST MODE

The composition for preventing and treating obesity and metabolic bone diseases according to the present invention is characterized by including a Eupatorium spp. extract as an active ingredient.

The health functional food for preventing and ameliorating obesity and metabolic bone diseases according to the present invention is characterized by including a Eupatorium spp. extract as an active ingredient.

### Mode for Invention

Hereinafter, the present invention will be explained in detail with reference to the following examples, including experimental examples. However, these examples are provided for illustrative purposes only and are not to be construed to limit the disclosure of the invention.

### Preparative Example 1

### Preparation of Extracts from Different Sections of Eupatorium japonicum

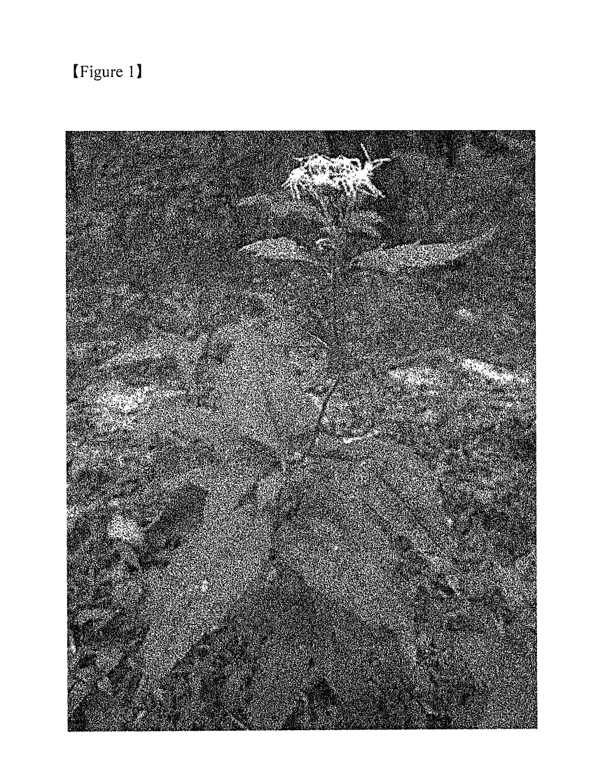

Eupatorium japonicum belonging to Eupatorium spp. was directly harvested from Mt. Gamak, Yangju-si, Kyeonggi-do, Korea, in September (FIG. 1).

The whole plant and different sections (flowers, leaves, and stems) of Eupatorium japonicum were completely dried at room temperature for 2 days and finely ground to obtain Eupatorium japonicum powder samples. 99.9% (v/v) methanol was added to each powder sample. 200 ml of the methanol was used per 10 g of the sample. The mixture was extracted in a shaking incubator at 120 rpm and 40° C. for 24 hours. The supernatant was filtered through a Whatman No. 1 filter paper. To the filtered Eupatorium japonicum sample was added 99.9% methanol. 200 ml of the methanol was used per 10 g of the filtered sample. The mixture was extracted for 24 hours and filtered in the same manner as described above. The filtrate was concentrated using a rotary vacuum evaporator under reduced pressure at 45° C. to completely remove the solvent, affording a methanol extract from the corresponding section of Eupatorium japonicum.

### Experimental Example 1

### Measurement of Stimulatory Effects of the Extracts from Different Sections of Eupatorium japonicum on Osteoblast Differentiation in C3H10T1/2 Cells

### (1) Alkaline Phosphatase (ALP) Staining

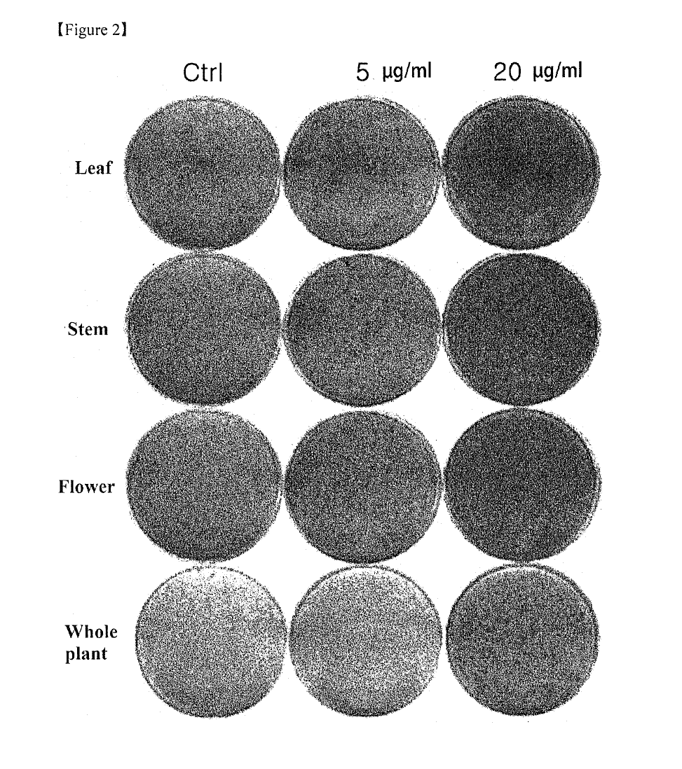

C3H10T1/2 cell line originating from mouse embryo fibroblasts is a pluripotent stem cell line that can be generally differentiated into various cell lineages, including osteoblasts and adipocytes. C3H10T1/2 cell line was cultured in DMEM medium supplemented with 10% FBS, 1% penicillin, and streptomycin at 37° C. and 5% CO₂. Cells were cultured to a concentration of 2.5×10⁴/ml, together with media containing 10 mM glycerophosphate and 50 μg/ml ascorbic acid for osteocyte differentiation, in a 6-well plate. The media were replaced with fresh ones every 3 day. The cultured cells were allowed to differentiate, together with 20 μg/ml and 40 μg/ml each of the extracts from Eupatorium japonicum, for a total of 9 days, followed by ALP staining using 5-bromo-4-chloro-3-indolyl phosphate/nitro blue tetrazolium (BCIO/NBT). The ALP staining results are shown in FIG. 2.

As a result, the ALP activities of the whole plant, leaf, stem, and flower extracts increased in a concentration-dependent manner. The leaf extract showed the highest ALP activity.

### (2) Analysis of Expression Levels of Osteoblast Differentiation Factors by Realtime RT-PCR

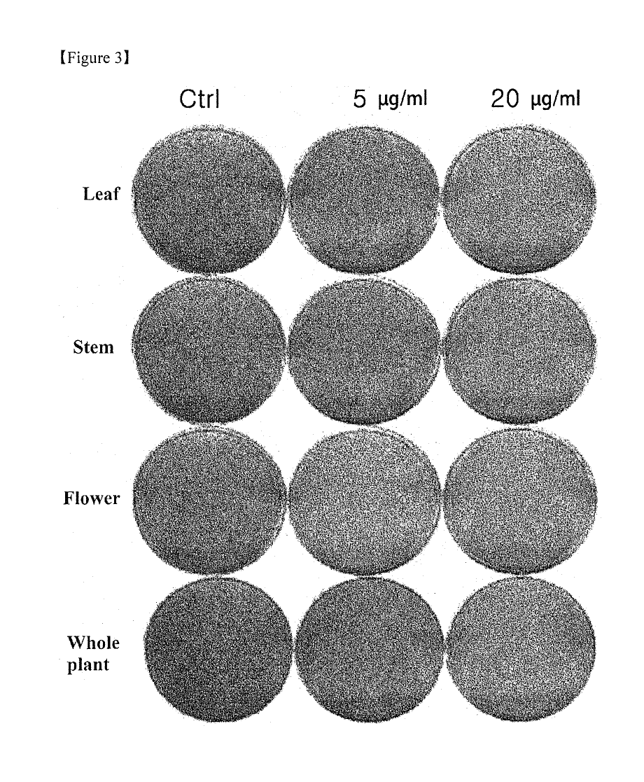

C3H10T1/2 cells were treated with 5 μg/ml and 20 μg/ml each of the extracts from Eupatorium japonicum for a total of 9 days while replacing media with fresh ones every 3 day. Thereafter, the mRNA expression levels of ALP, osterix, and CO1 I as important factors for osteoblast formation were determined by realtime RT-PCR, and the results are shown in FIGS. 3-6.

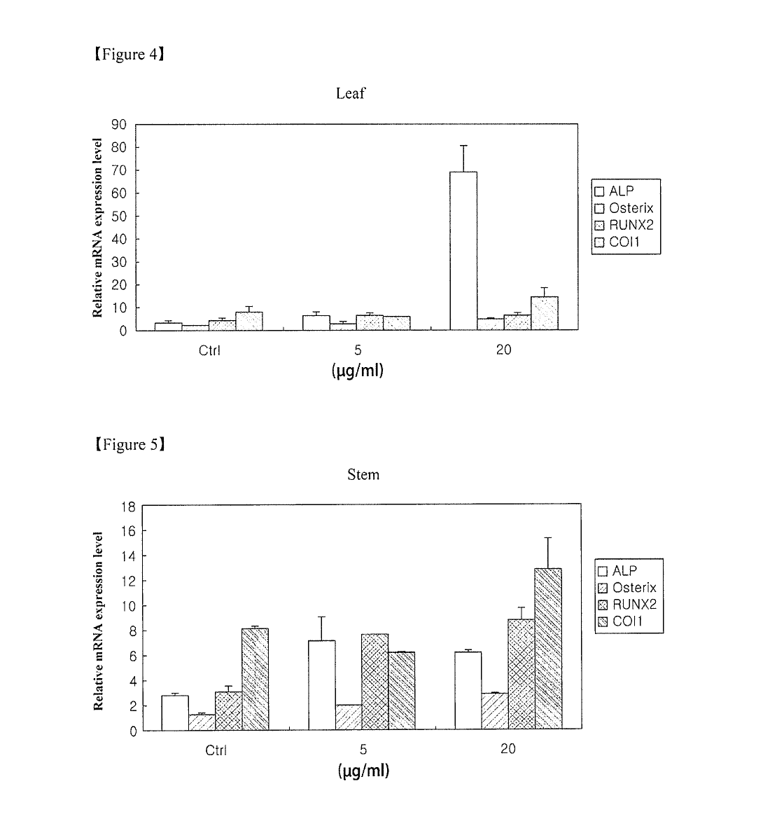

As a result, the whole plant extract at a concentration of 5 μg/ml increased the mRNA expression level of osterix among osteoblast-related differentiation genes compared to control group (ctrl), and the whole plant extract at a concentration of 20 μg/ml increased the mRNA expression levels of ALP and CO1 I compared to control group (ctrl) (FIG. 3). No significant difference in mRNA expression level was observed between the group treated with the leaf extract at a concentration of 5 μg/ml and control group but a significantly high mRNA expression level of ALP was observed in the group treated with the leaf extract at a concentration of 20 μg/ml compared to in control group (FIG. 4).

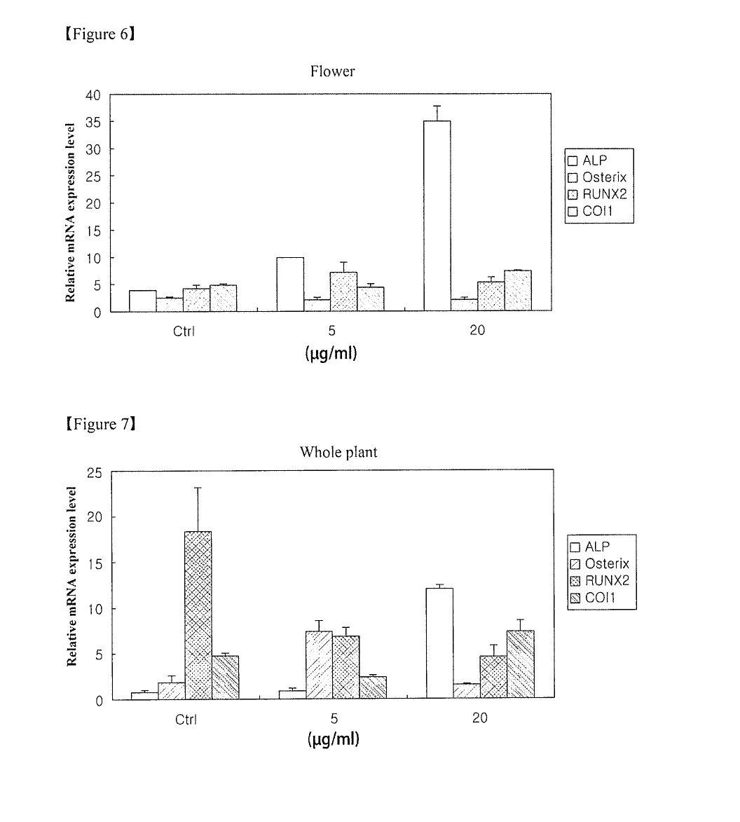

A high mRNA expression level of ALP was observed in the group treated with the stem extract at a concentration of 5 μg/ml and high mRNA expression levels of ALP and CO1 I were observed in the group treated with the stem extract at a concentration of 20 μg/ml compared to in control group (FIG. 5). A high mRNA expression level of ALP was observed in the group treated with the flower extract at a concentration of 20 μg/ml compared to in control group (FIG. 6).

### Experimental Example 2

### Measurement of Inhibitory Effects of the Extracts from Different Sections of Eupatorium japonicum on Adipocyte Differentiation in C3H10T1/2 Cells

### (1) Measurement of Inhibition of Adipocyte Differentiation by Oil Red O Staining

C3H10T1/2 cells were cultured to a concentration of 2.5×10⁴/ml, together with media containing 1 μM dexamethasone, 5 ng/ml insulin, and 20 nM PPARγ for adipocyte differentiation and 5 μg/ml and 20 μg/ml each of the extracts from Eupatorium japonicum, for 9 days. After removal of the media, the cells were fixed in 4% formaldehyde and stained with 0.5% Oil red O. The results are shown in FIG. 7.

The photographs of FIG. 7 show that all of the whole plant, leaf, stem, and flower extracts inhibited the differentiation of adipocytes in a concentration-dependent manner. Particularly, the flower extract showed the highest inhibitory effect on adipocyte differentiation.

### (2) Analysis of Expression Levels of Adipocyte Differentiation Factors by Realtime RT-PCR

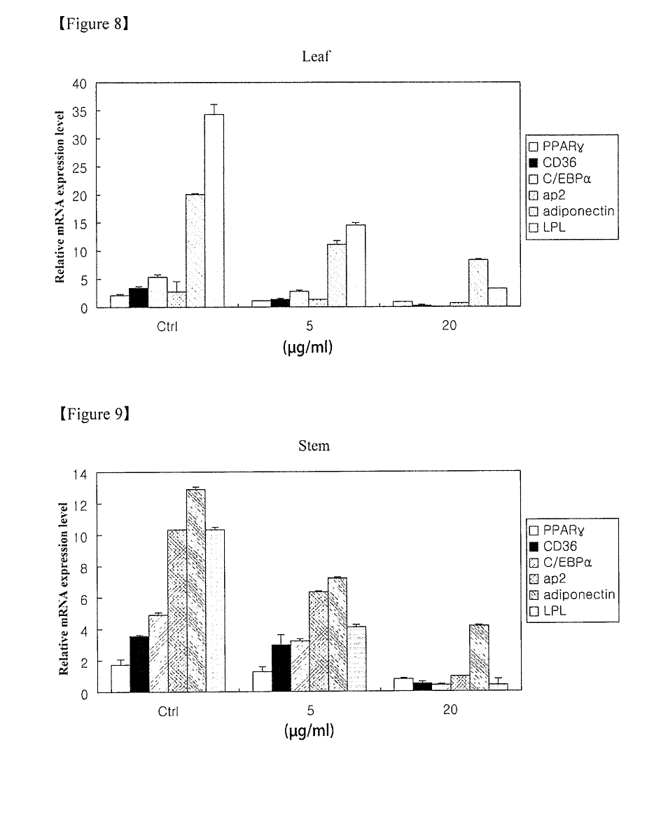

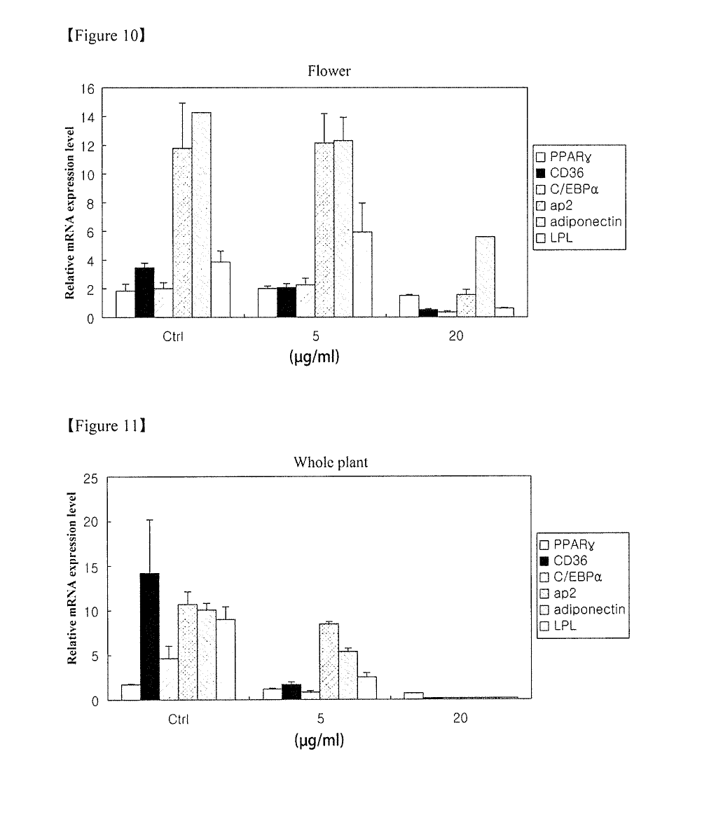

C3H10T1/2 cells were treated with the whole plant, leaf, stem, and flower extracts for a total of 10 days in media containing substances for adipocyte differentiation, and the relative mRNA expression levels of PPARγ, AP2, CD36, adiponectin, C/EBPα, and LPL as important factors for adipocyte formation were determined by realtime RT-PCR. The results are shown in FIGS. 8-11.

The results demonstrate that all of the whole plant, leaf, stem, and flower extracts inhibited the activities of PPARγ, AP2, CD36, adiponectin, C/EBPα, and LPL in a concentration-dependent manner.

### Preparative Example 2

### Preparation of Extracts from Eupatorium japonicum Harvested at Different Time Periods

The stems of Eupatorium japonicum were harvested from Mt. Gamak located in Yangju-si, Kyeonggi-do, Korea, on the same date of each month from May to September, completely dried at room temperature for 2 days, and finely ground to obtain Eupatorium japonicum powder samples. 99.9% (v/v) methanol was added to each powder sample. 200 ml of the methanol was used per 10 g of the sample. The mixture was extracted in a shaking incubator at 120 rpm and 40° C. for 24 hours. The supernatant was filtered through a Whatman No. 1 filter paper. To the filtered Eupatorium japonicum sample was added 99.9% methanol. 200 ml of the methanol was used per 10 g of the filtered sample. The mixture was extracted for 24 hours and filtered in the same manner as described above. The filtrate was concentrated using a rotary vacuum evaporator under reduced pressure at 45° C. to completely remove the solvent, affording a methanol extract from the stems of Eupatorium japonicum harvested at the corresponding time period.

### Preparative Example 3

### Preparation of Solvent Fractions from the Eupatorium japonicum Extracts

The methanol extract from the stems of Eupatorium japonicum harvested in September was fractionated stepwise with solvents of different polarties. First, the methanol extract was fractionated with hexane. For the hexane fractionation, the methanol extract, hexane, and water were mixed in a ratio of 1:20:20. Concentration of the mixture gave a hexane fraction.

The aqueous fraction was fractionated with dichloromethane, ethyl acetate, and butanol in separatory funnels to obtain dichloromethane, ethyl acetate, butanol, and aqueous fractions. The fractions were concentrated, freeze-dried, and stored before use.

### Experimental Example 3

### Measurement of Stimulatory Effects of the Extracts from Eupatorium japonicum Harvested at Different Time Periods and the Solvent Fractions on Osteoblast Differentiation in C3H10T1/2 Cells and Primary Mesenchymal Stem Cells

### (1) Alkaline Phosphatase (ALP) Staining

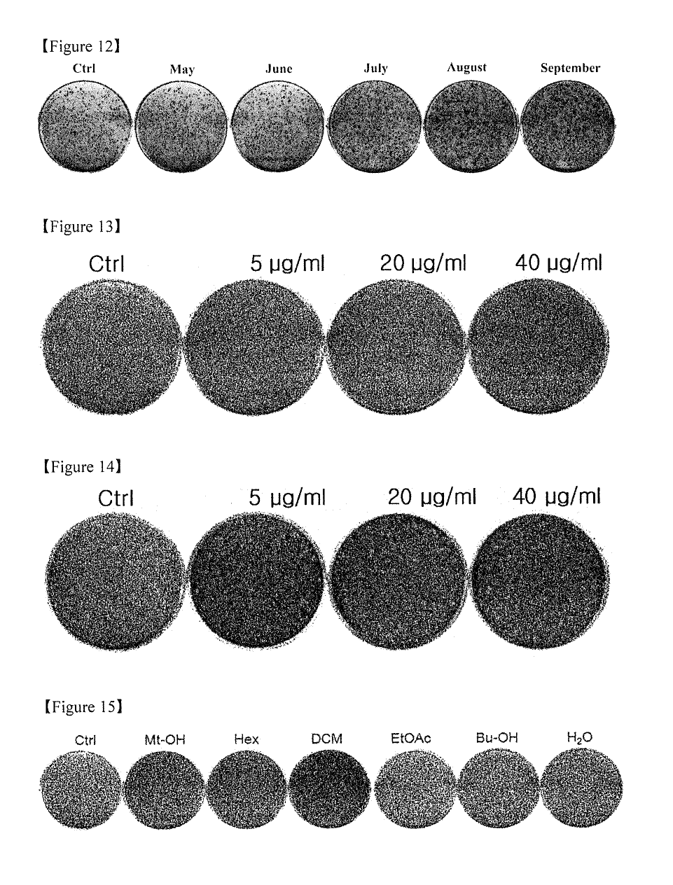

The procedure of Experimental Example 1 (1) was repeated except that the methanol extracts of Preparative Example 2 and the solvent fractions of Preparative Example 3 were used as samples. The ALP staining results are shown in FIGS. 12, 13, 14, 15, and 16.

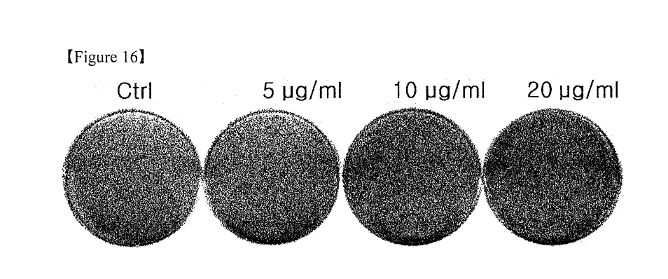

As a result, the extract from the stems of Eupatorium japonicum harvested in September showed the highest ALP activity (FIG. 12) and the dichloromethane (DCM) fraction showed higher ALP activity than the other solvent fractions (FIG. 15).

As a result of the treatment with the DCM fraction at concentrations of 5 μg/ml, 10 μg/ml, and 20 μg/ml, the ALP activity of the DCM fraction increased in a concentration-dependent manner (FIG. 16).

### (2) Analysis of Expression Levels of Osteoblast Differentiation Factors by Realtime RT-PCR

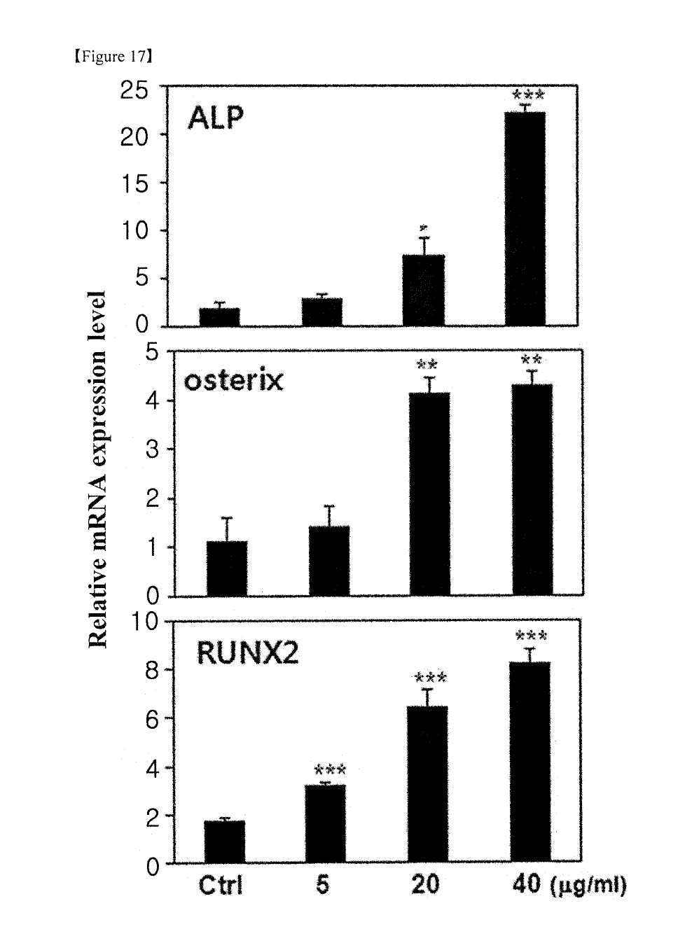

C3H10T1/2 cells and primary mesenchymal stem cells were treated with 5 μg/ml, 20 μg/ml, and 40 μg/ml each of the stem extracts from Eupatorium japonicum and the six solvent fractions for a total of 9 days while replacing media with fresh ones every 3 day. Thereafter, the mRNA expression levels of ALP, osterix, and RUNX2 as important factors for osteoblast formation were determined by realtime RT-PCR, and the results are shown in FIGS. 17, 18, and 19.

As a result, high mRNA expressions of ALP and osterix among osteoblast differentiation-related genes in C3H10T1/2 cells were observed in the groups treated with the extract from the stems harvested in September at concentrations of 20 μg/ml and 40 μg/ml compared to in control group (ctrl), and high mRNA expressions of RUNX2 were observed in the groups treated with the stem extract at concentrations of 5 μg/ml, 20 μg/ml, and 40 μg/ml compared to control group (ctrl) (FIG. 17).

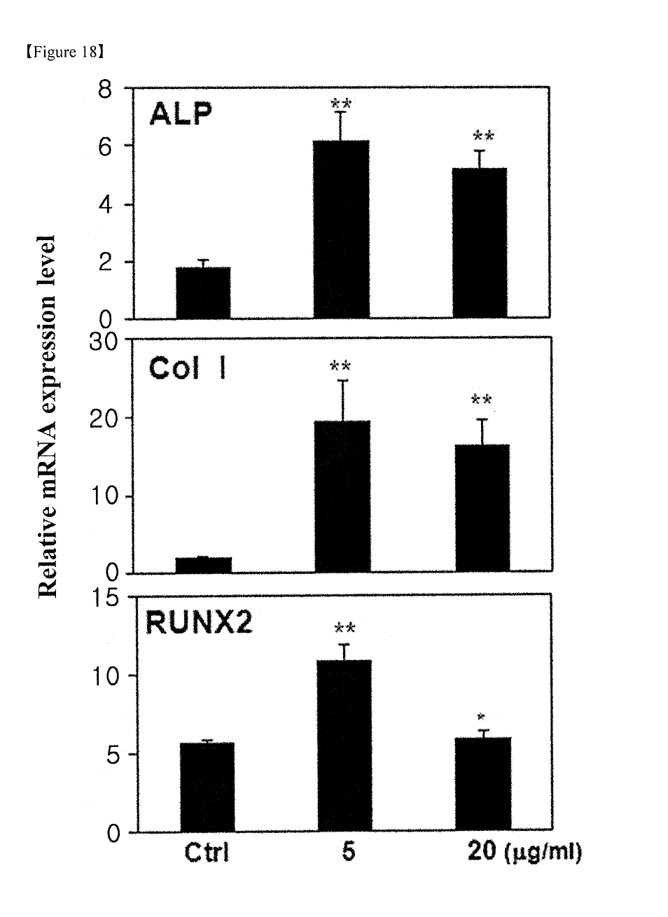

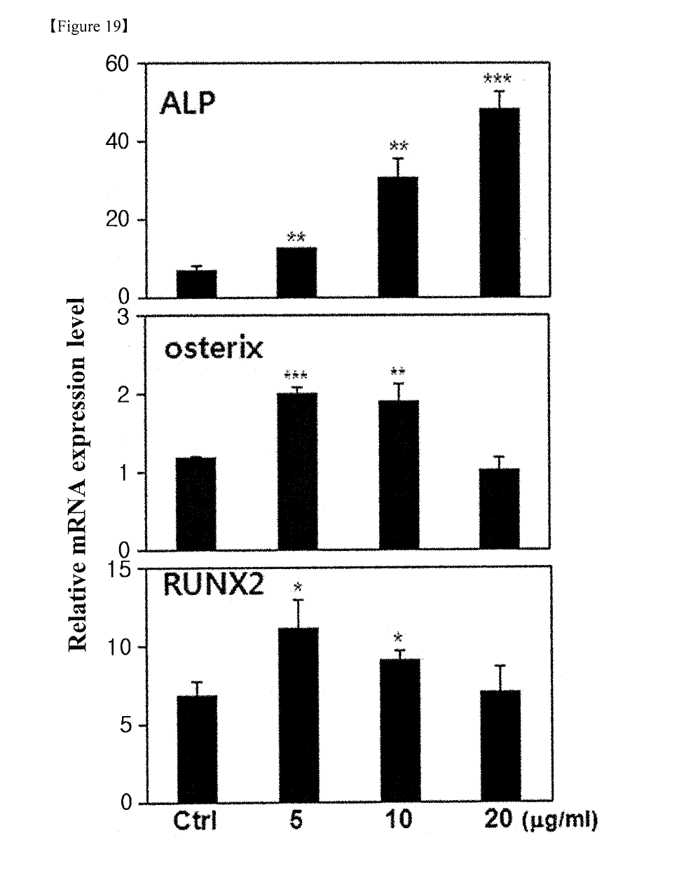

High mRNA expressions of ALP and osterix among osteoblast differentiation-related genes in primary mesenchymal stem cells were observed in the groups treated with the extract from the stems harvested in September at concentrations of 20 μg/ml and 40 μg/ml compared to in control group (ctrl), and high mRNA expressions of ALP, Co1 I, and RUNX2 were observed in the groups treated with the stem extract at concentrations of 5 μg/ml and 20 μg/ml compared to in control group (ctrl) (FIG. 18).

High mRNA expressions of ALP among osteoblast differentiation-related genes in C3H10T1/2 cells were observed in the groups treated with the DCM fraction from the extract from the stems harvested in September at concentrations of 5 μg/ml, 10 μg/ml, and 20 μg/ml compared to in control group, and high mRNA expressions of osterix and RUNX2 were observed in the groups treated with the DCM fraction at concentrations of 5 μg/ml and 10 μg/ml compared to in control group (ctrl) (FIG. 19).

### Experimental Example 4

### Measurement of Inhibitory Effects of the Extracts from Eupatorium japonicum Harvested at Different Time Periods and the Solvent Fractions on Adipocyte Differentiation in C3H10T1/2 Cells and Primary Mesenchymal Stem Cells

### (1) Measurement of Inhibition of Adipocyte Differentiation by Oil Red O Staining

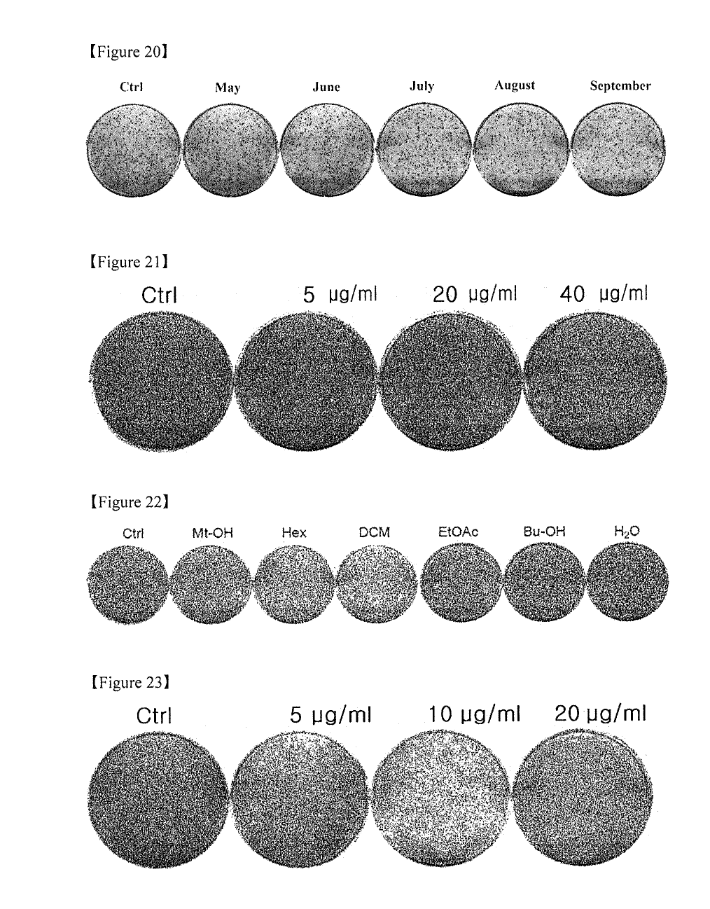

C3H10T1/2 cells were allowed to differentiate to a concentration of 2.5×10⁴/ml, together with media containing 1 μM dexamethasone, 5 μg/ml insulin, and 20 nM PPARγ for adipocyte differentiation, 5 μg/ml, 20 μg/ml, and 40 μg/ml each of the extracts from the stems of Eupatorium japonicum, and the six solvent fractions, for a total of 9 days. After removal of the media, the cells were fixed in 4% formaldehyde and stained with 0.5% Oil red O. The results are shown in FIGS. 20, 21, 22, and 23.

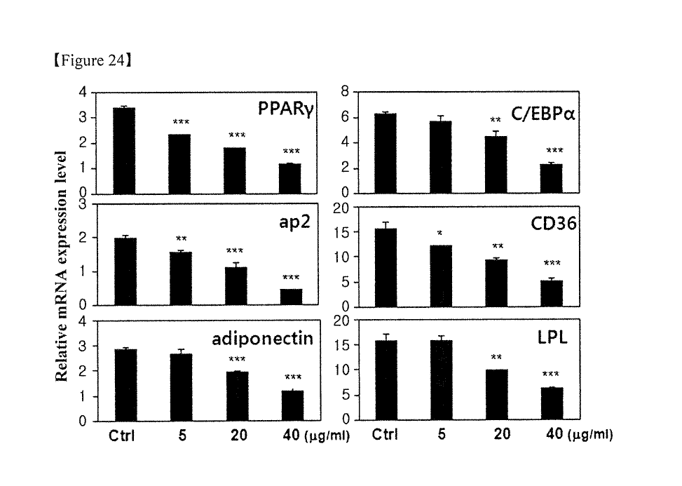

As a result, the extract from the stems of Eupatorium japonicum harvested in September had the highest inhibitory effect on adipocyte differentiation among the extracts from the stems of Eupatorium japonicum harvested in the different months (FIG. 20), and the dichloromethane (DCM) fraction had the highest inhibitory effect on adipocyte differentiation among the six solvent fractions (FIG. 22). As a result of the treatment with the DCM fraction at concentrations of 5 μg/ml, 10 μg/ml, and 20 μg/ml, the adipocyte differentiation was inhibited in a concentration-dependent manner (FIG. 23).

### (2) Analysis of Expression Levels of Adipocyte Differentiation Factors by Realtime RT-PCR

C3H10T1/2 cells and primary mesenchymal stem cells were treated with 5 μg/ml, 20 μg/ml, and 40 μg/ml each of the stem extracts from Eupatorium japonicum and the six solvent fractions for a total of 9 days while replacing media with fresh ones every 3 day. Thereafter, the mRNA expression levels of ALP, osterix, and RUNX2 as important factors for adipocyte formation were determined by realtime RT-PCR, and the results are shown in FIGS. 24, 25, and 26.

As a result, low mRNA expressions of PPARγ, AP2, and CD36 among adipocyte differentiation-related genes in C3H10T1/2 cells were observed in the groups treated with the extract from the stems harvested in September at concentrations of 5 μg/ml, 20 μg/ml and 40 μg/ml compared to control group (ctrl), and low mRNA expressions of adiponectin, C/EBPα and LPL were observed in the groups treated with the stem extract at concentrations of 20 μg/ml and 40 μg/ml compared to control group (ctrl) (FIG. 24).

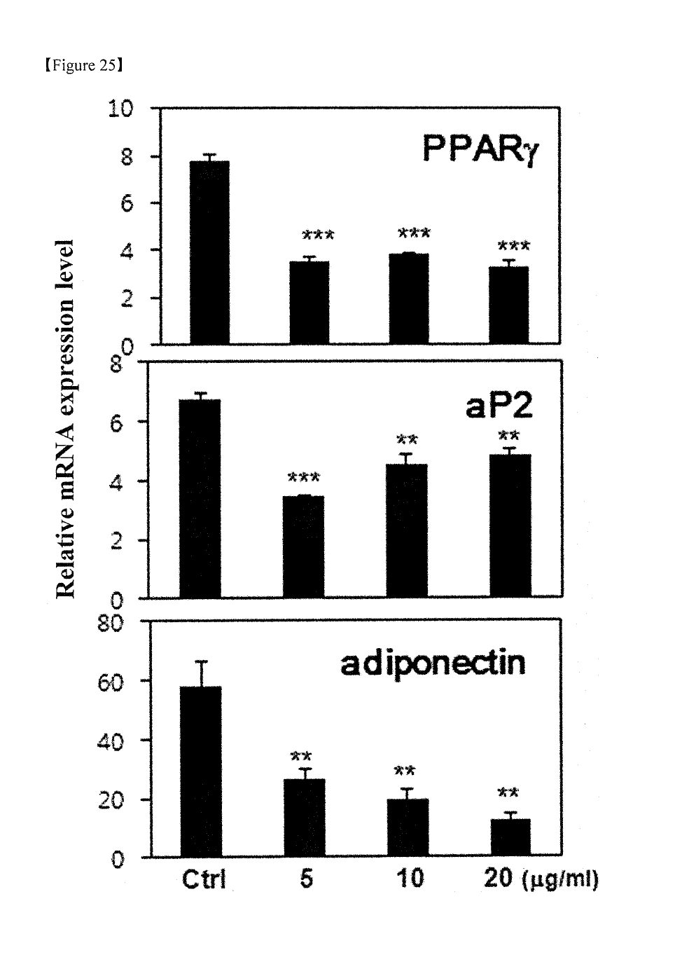

Low mRNA expressions of PPARγ, AP2, and adiponectin among adipocyte differentiation-related genes in primary mesenchymal stem cells were observed in the groups treated with the extract from the stems harvested in September at concentrations of 5 μg/ml, 20 μg/ml and 40 μg/ml compared to control group (ctrl) (FIG. 25).

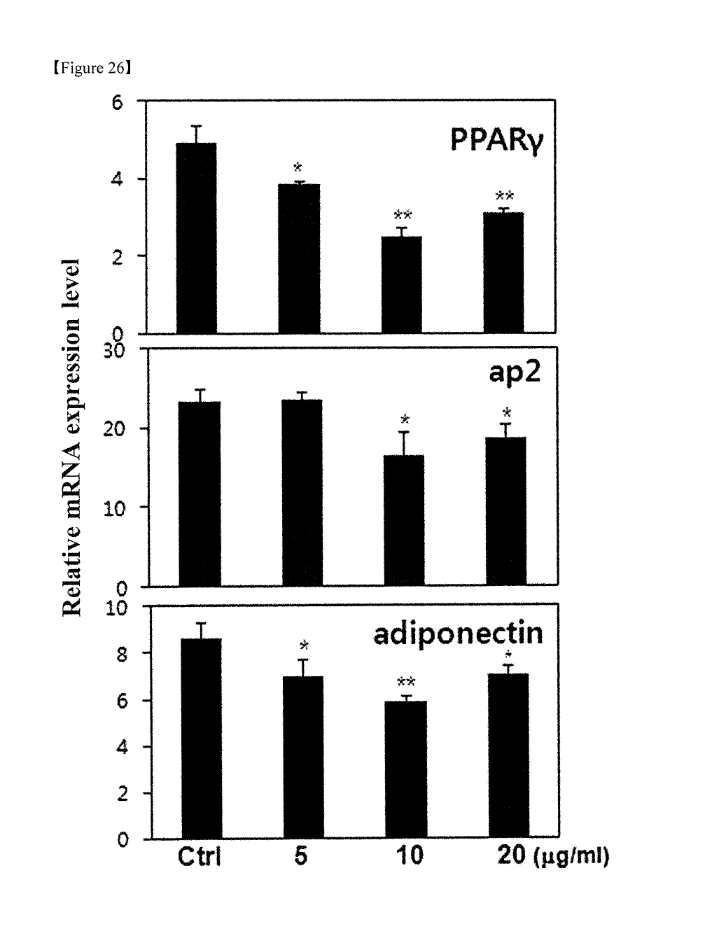

Low mRNA expressions of PPARγ and adiponectin among adipocyte differentiation-related genes in C3H10T1/2 cells were observed in the groups treated with the DCM fraction from the extract from the stems harvested in September at concentrations of 5 μg/ml, 10 μg/ml, and 20 μg/ml compared to control group (ctrl), and low mRNA expressions of Apt were observed in the groups treated with the DCM fraction at concentrations of 10 μg/ml and 20 μg/ml compared to control group (ctrl) (FIG. 26).

### Experimental Example 5

### Bone Mineral Density (BMD) and Histological Analysis in Animal Model with Osteoporosis Caused by Ovariectomy

### (1) Breeding and Ovariectomy of Experimental Animals

11-week-old female SD rats were purchased from Daehan Biolink (Korea) and acclimated for a period of one week. Ovariectomy was performed at the age of 12 weeks, and the rats were allowed to recover for one week after surgery. The experimental animals were bred in cages, one in each cage, and they were kept under the following environmental conditions: 24±2° C. indoor temperature, 55±10% relative humidity, and 12-hr light-dark cycle. Free access to food and water was available.

The experimental animals were divided into three groups, 10 animals per group. The first group (sham) consisted of 10 non-ovariectomized animals, the second group (ovx-control) consisted of 10 ovariectomized animals, and the third group (ovx+SEE 50 mg/kg) consisted of 10 ovariectomized animals received 50 mg/kg of the extract from the stems of Eupatorium japonicum harvested in September. Ovariectomy was performed in such a manner that each animal was anesthetized with zoletil and rompun, the skin was shaved and incised, the ovaries were removed, and the wound was closed with sutures. The methanol extract from the stems of Eupatorium japonicum harvested in September (Preparative Example 2) was used as a sample. The sample was administered orally for a total of 6 weeks and the body weights of the animals were measured daily.

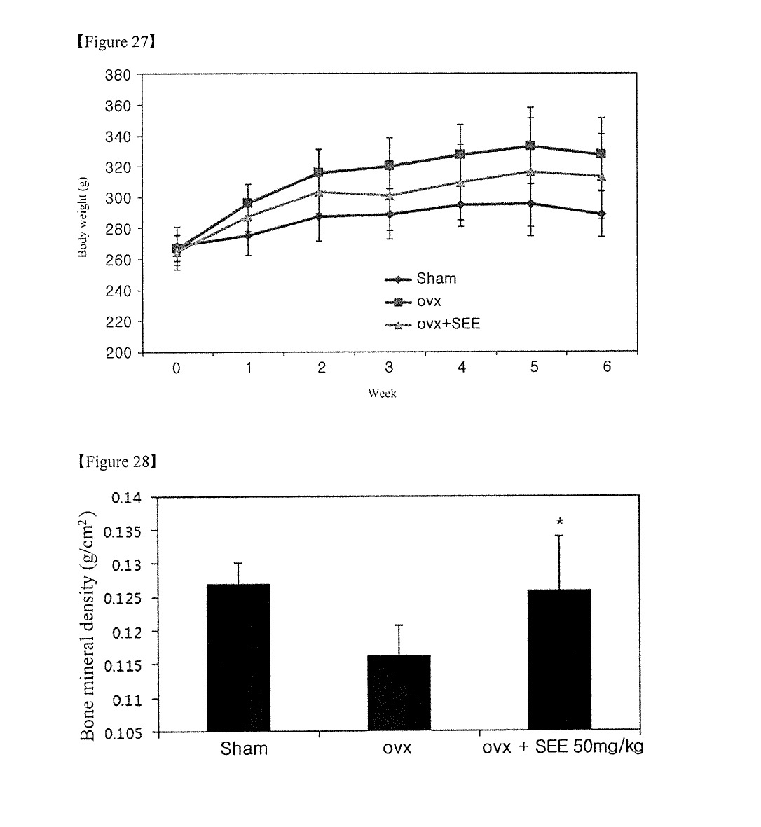

As a result, the ovariectomized group (ovx) having undergone a reduction in estrogen level after ovariectomy showed a tendency toward weight gain compared to the non-ovariectomized group (sham), and the group received the extract from the stems of Eupatorium japonicum (ovx+SEE 50 mg/kg) showed a tendency toward weight loss compared to the ovariectomized group (ovx-control) (FIG. 27).

### (2) Bone Mineral Density (BMD) Measurement

The femurs and tibias of the experimental animals were removed at the age of 19 weeks. The bone mineral densities of the femurs were measured using pDEXA (Forearm: X-Ray, NORLAND, Bone Densitometer, USA).

As a result, the ovariectomized group (ovx-control) showed a significant decrease in bone mineral density (BMD) compared to the non-ovariectomized group (sham). The ovariectomized group received the extract from the stems of Eupatorium japonicum (ovx+SEE 50 mg/kg) showed a significant increase in bone mineral density (BMD) compared to the ovariectomized group (ovx-control) (FIG. 28).

### (3) Histological Analysis

### H&E Staining

The tibia removed from each animal was fixed in 10% formaldehyde and was then subjected to decalcification to produce paraffin blocks. The paraffin blocks were sliced to a thickness of 5 μm. The slices were stained with hematoxylin & eosin (H&E).

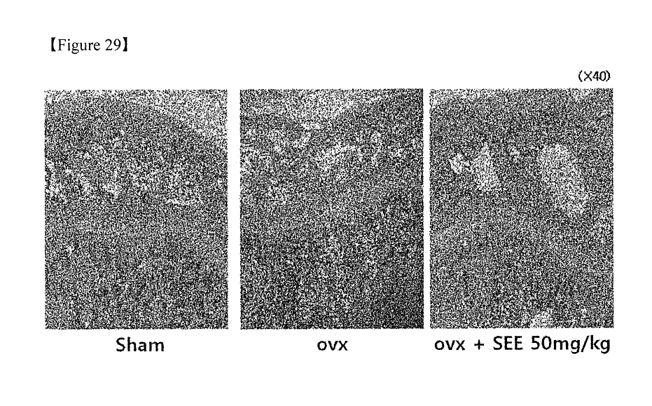

As a result, the ovariectomized group (ovx+control) was observed to have an increased amount of adipocytes in the bone marrow compared to the non-ovariectomized group (sham), and the group received the extract from the stems of Eupatorium japonicum (ovx+SEE 50 mg/kg) was observed to have a reduced amount of adipocytes in the bone marrow compared to the ovariectomized group (ovx+control) (FIG. 29).

The compositions containing the extracts according to the present invention will be explained with reference to the following formulation examples. These examples do not serve to limit the invention and are merely intended to provide a detailed explanation of the invention.

### Formulation Example 1

### Production of Powder Preparations

| | | |
|---|---|---|
| The whole plant extract | 20 | mg |
| (Preparative Example 1) | | |
| Lactose | 100 | mg |
| Talc | 10 | mg |

The ingredients were mixed and filled in airtight bags to produce powder preparations.

### Formulation Example 2

### Production of Tablet Preparations

| | | |
|---|---|---|
| The extract from the stems harvested | 10 | mg |
| in September (Preparative Example 2) | | |
| Corn starch | 100 | mg |
| Lactose | 100 | mg |
| Magnesium stearate | 2 | mg |

The ingredients were mixed and compressed to produce tablet preparations in accordance with a suitable method known in the art.

### Formulation Example 3

### Production of Capsule Preparations

| | | |
|---|---|---|
| The extract from the stems harvested | 10 | mg |
| in September (Preparative Example 2) | | |
| Crystalline cellulose | 3 | mg |
| Lactose | 14.8 | mg |
| Magnesium stearate | 0.2 | mg |

The ingredients were mixed and filled in gelatin capsules to produce capsule preparations in accordance with a suitable method known in the art.

### Formulation Example 4

### Preparation of Injectable Preparations

| | | |
|---|---|---|
| The dichloromethane fraction | 10 | mg |
| (Preparative Example 3) | | |
| Mannitol | 180 | mg |
| Sterile distilled water for injection | 2974 | mg |
| Na₂HPO₄•12H₂O | 26 | mg |

In accordance with a suitable method known in the art, the ingredients were mixed and filled in ampoules (2 ml per ampoule) to produce injectable preparations.

### Formulation Example 5

### Production of Liquid Preparations

| | | |
|---|---|---|
| The whole plant extract | 20 | mg |
| (Preparative Example 1) | | |
| Isomerized glucose syrup | 10 | g |
| Mannitol | 5 | g |

| | |
|---|---|
| Purified water | Appr. amount |

In accordance with a suitable method known in the art, the ingredients were dissolved in purified water, an appropriate amount of lemon flavor was added to the solution, the volume was adjusted to a total of 100 ml with purified water, and the mixture was filled in amber glass bottles and sterilized to produce liquid preparations.

### Formulation Example 6

### Production of Health Food

| The whole plant extract | 1,000 | mg |
|---|---|---|
| (Preparative Example 1) | | |
| Vitamin mixture | Appr. amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Mineral mixture | Appr. amount | |
| Ferrous sulfate | 1.75 | mg |
| Zinc oxide | 0.82 | mg |
| Magnesium carbonate | 25.3 | mg |
| Potassium phosphate | 15 | mg |
| Calcium phosphate | 55 | mg |
| Potassium citrate | 90 | mg |
| Calcium carbonate | 100 | mg |
| Magnesium chloride | 24.8 | mg |

Although the vitamins and the mineral mixture were selected from ingredients relatively suitable for health foods and were mixed in a preferable ratio, it should be understood that the composition may be appropriately changed and modified. For example, in accordance with a known method for producing a health food, the ingredients are mixed, the mixture is formulated into granules, and the granules are used to prepare a health food composition.

### Formulation Example 7

### Production of Health Beverage

| | | |
|---|---|---|
| The extract from the stems harvested | 1,000 | mg |
| in September (Preparative Example 2) | | |
| Citric acid | 1,000 | mg |
| Oligosaccharide | 100 | g |
| Japanese apricot concentrate | 2 | g |
| Taurine | 1 | g |

| | |
|---|---|
| Purified water | up to a total |
| | of 900 ml |

In accordance with a known method for producing a health beverage, the ingredients were mixed, heated with stirring at 85° C. for about 1 hour, filtered, filled in a 2 L sterile container, sealed, sterilized, and refrigerated. The solution was used to prepare a health beverage composition.

### INDUSTRIAL APPLICABILITY

Although the compositions were prepared by mixing the ingredients relatively suitable for favorite beverages in preferable ratios, it should be understood that the compositions may be appropriately changed and modified depending on regional and national preferences, such as demand classes, demand countries, and applications of use.

1. A composition for preventing and treating obesity and metabolic bone diseases, comprising as an active ingredient, an extract from a Eupatorium spp. plant.

2. The composition according to claim 1, wherein the composition has a stimulatory activity on osteoblast differentiation and an inhibitory activity on adipocyte differentiation.

3. The composition according to claim 2, wherein the Eupatorium spp. plant is selected from E. japonicum, E. lindleyanum, E. makinoi var. oppisitifolium, E. rugosum, and closely related plants belonging to the genus Eupatorium.

4. The composition according to claim 3, wherein the extract from the Eupatorium spp. plant is prepared from the whole plant, leaves, stems or flowers of Eupatorium japonicum.

5. The composition according to claim 4, wherein the Eupatorium japonicum is harvested from July to September.

6. The composition according to claim 2, wherein the extract is prepared by dissolving the Eupatorium spp. plant in a solvent selected from water, comprising purified water, a C₁-C₄ lower alcohol, a non-polar solvent, and a mixed solvent thereof.

7. The composition according to claim 6, wherein the solvent is a 5% to 100% (v/v) aqueous alcoholic solution of water and a C₁-C₄ lower alcohol or pure alcohol.

8. The composition according to claim 6, wherein the non-polar solvent is dichloromethane, chloroform, diethyl ether, ethyl acetate, hexane, or a supercritical fluid.

9. The composition according to claim 7, wherein the solvent is a 70 to 99.9% (v/v) aqueous methanolic or ethanolic solution.

10. The composition according to claim 9, wherein the extract is a hexane fraction obtained by extraction with the 70 to 99.9% (v/v) aqueous methanolic or ethanolic solution and fractionation with hexane.

11. The composition according to claim 10, wherein the extract is a dichloromethane fraction obtained by fractionation of the hexane fraction with dichloromethane.

12. A health functional food for preventing and ameliorating obesity and metabolic bone diseases, comprising a Eupatorium spp. extract as an active ingredient.

13. The health functional food according to claim 12, wherein the health functional food takes the form of a powder, a granule, a tablet, a capsule, or a beverage.
